(19) United States
(12) Patent Application Publication
(10) Pub. No.: US 2003/0014067 A1
(43) Pub. Date: Jan. 16, 2003

(54) Surgical saw blade

(76) Inventors: Michael Kullmer, Lemgo (DE); Andreas Koster, Lemgo (DE); Matthias Gasser, Lemgo (DE); Volker Brinkmann, Lemgo (DE)

Correspondence Address: THOMPSON COBURN, LLP, ONE FIRSTAR PLAZA, SUITE 3500, ST LOUIS, MO, 63101, US

(21) Appl. No.: 10/179,378

(22) Filed: Jun. 25, 2002

(30) Foreign Application Priority Data
Jun. 25, 2001 (DE) 101 31 023.4
Jun. 25, 2001 (DE) 101 30 007.7

(51) Int. Cl. A61B 17/14

(52) U.S. Cl. 606/176; 606/178

(57) ABSTRACT

The invention relates to a surgical saw blade having a flat, plate-shaped body (1) being provided with a toothing (2) at least one edge portion, said toothing comprising a plurality of uncrossed teeth, said teeth each consisting of groups comprising three teeth (3, 4, 5) of different shape, or wherein the toothing (2) comprises pairs (5, 6) of two teeth (3, 4) being arranged symmetrically to each other in relation to a plane perpendicular to the center plane of the body (1).

### BACKGROUND OF THE INVENTION

The present invention relates to a surgical saw blade comprising a clamping portion and a working area provided with a toothing.

Surgical saw blades of the described type are known from the prior art in many different designs. They are typically mounted on a drive unit by which they can be induced to perform an oscillating reciprocating movement. It is thereby possible to carry out, for instance, precise bone cuts. The saw blades can be guided manually, but it is also possible to use the blades together with templates to produce precise predetermined cuts.

When using such surgical saw blades, it is important that the cuts are carried out precisely at the correct position and that the surrounding bone is not damaged more than absolutely necessary.

The state of the art discloses many different designs of saw blades being provided with crossed teeth. However, such crossed saw blades are not sufficiently precise and have the disadvantage that the saw blade becomes untrue during cutting and the cut in this way becomes unprecise.

From U.S. Pat. No. 5,306,285, a surgical saw blade is known, comprising non-crossed teeth. The teeth are alternatingly from different sides ground or chamfered, respectively, and additionally feature a channel grinding on one side. The channel grindings are formed alternatingly on the one or other surface of the body of the saw blade. In contrast, crossed saw blades, which have a very agitated behaviour when in use and prevent a well-directed non-traumatic sawing, the saw blades according to the aforementioned US patent are somewhat better, but still behave very agitated in practical use.

### BRIEF SUMMARY OF THE INVENTION

It is an object of the invention to provide a surgical saw blade of the aforementioned kind, which shows an optimised cutting behaviour and permits a precise cutting without considerable damages to the surrounding bone material while being of a simple structure and producible at low costs.

According to the invention, the object is achieved by the features of the main independent claims, the dependent claims showing further advantageous embodiments of the invention.

The inventive saw blade stands out due to a series of substantive advantages.

Thus, according to the invention, it is provided in a first embodiment that the teeth each are formed of groups, which groups each comprise three differently formed teeth. The complete toothing therefore consists of a sequence of three teeth each, which belong to one group. Due to this sequence of different teeth, it is possible to specifically influence the saw behavior. Vibrations and chatter marks are thus prevented. The inventive toothing as a whole stands out due to its very good centering behaviour and precise guidability. Therefore, the cutting of the bone can be carried out very precisely, leading to considerable advantages for the subsequent surgical actions.

According to the invention, it is provided in a second embodiment that each toothing comprises pairs of two teeth which are formed symmetrically to each other relative to a plane perpendicular to the intermediate plane of the body. By way of this symmetrical formation of the teeth, the power entry into the toothing becomes more equal, so that the toothing can be better centered, resulting in a very smooth cutting procedure. Thus, it is possible to guide the saw blade with greater precision and to generate more precise bone cuttings.

The mentioned advantages exist for a freehand use of the saw blade as well as for use in specifically provided templates.

As the saw blade performs an oscillating reciprocating movement, the single teeth of the groups or pairs are employed regularly, which results in an excellent regular chip removal.

In a particularly advantageous embodiment of the first aspect of the invention it is provided that the intermediate tooth of the group comprises a lance. This embodiment results in a particularly good centering of the saw blade.

Furthermore, it may be advantageous if the intermediate tooth of the group relative to the intermediate plane of the body is formed symmetrically. Thus, the tooth may be provided with a channel grinding at both lateral sides, due to which the centering effect as well as the chip removal are ameliorated.

The two lateral teeth of the group may each be provided with a cross cutting. In this way, smaller chips are generated during the oscillating use, which furthermore may be discharged through the channels formed by the channel grinding. Thus, it results that the formation of channels on a lateral side of each first or last tooth of the group, respectively, is especially advantageous. Herein, it is also particularly advantageous if the two lateral teeth with respect to the channel grinding are formed towards different sides of the saw blade. Thus, a changing of the teeth shapes used at a bone position occurs alternatingly during the saw process. This results in a smooth, equally wide cut, which is generated subsequently by the different teeth.

It is particularly advantageous if, as described, the channel grinding is placed at one of the teeth on the left side and at the other one on the right side. It is preferable that the channel grinding does not exceed the center of the respective tooth. Thus, the edges of the respective teeth comprise a cutting edge running obliquely up to the center.

According to the invention, it is possible to form all three teeth of one group to be equally high. However, it is also possible to form the intermediate tooth to be higher than the two lateral teeth. In this way, a further amelioration of the centering effect of the toothing is achieved.

Especially favourable is the further development of the second aspect, in which each tooth is formed in the shape of a body establishing an equal-sided or oblique-angled triangle in side view, one edge of which is chamfered unilaterally. Thus, the cutting edges are used alternatingly towards different sides of the body of the saw blade. This results in a smooth cut and prevents chatter marks and the like.

The chamfers of each pair of teeth may be arranged preferably such that they point away from each other. In this way, the saw blade may be identically used in both cutting directions.

The inventive pairs of teeth are preferably arranged alternatingly with respect to the intermediate plane of the body of the saw blade. That means that “left” pairs of teeth are used first, followed by pairs of “right” teeth. Also in this way, the precision of the cutting as well as a smooth operation of the saw blade are increased.

It may be particularly preferable to arrange at least one additional tooth between two adjacent pairs of teeth. This may take place by additional, changed cutting geometries, in order to ameliorate the centering on the one hand and on the other hand to remove bone portions which cannot be processed in an optimal way by the two subsequent pairs of teeth.

The at least one additional tooth may have the same height as the adjacent pairs of teeth, however, it is also possible to provide a different height. It may for example be preferable to form the additional tooth higher or lower. It is furthermore possible to provide the additional tooth with a cross cutting, resulting in a different cutting geometry for this tooth.

In a further development of the invention, it is also possible to arrange two additional teeth between the adjacent pairs of teeth. In this case, it may be preferable if the tooth following the pair of teeth is identical to the subsequent tooth of the following pair of teeth. Also in this case, additional teeth between the single pairs may be used when using the saw blade in a reciprocating oscillating movement, thus contributing to a changed cutting geometry. In the last mentioned embodiment, no new cutting geometries are added, but only those tooth shapes repeated which are identical to the respective subsequent pair of teeth. Thus, the manufacturing of the saw blade is also greatly simplified.

### BRIEF DESCRIPTION OF THE SEVERAL VIEWS OF THE DRAWINGS

The invention shall now be explained with reference to embodiments taken in conjunction with the drawings, in which like parts are provided with like reference numerals. In the drawings:

FIG. 1 is a schematic side view of the inventive saw blade according to the first embodiment,

FIG. 2 is an enlarged partial view of the toothing area in a first manufacturing step,

FIG. 3 is the partial view of the toothing areas shown in FIG. 2, in which the height of the teeth has been modified,

FIG. 4 is an enlarged detailed view of section IV of the toothing area of FIG. 3,

FIG. 5 is an illustration of the completed inventive toothing,

FIG. 6 is an enlarged detailed illustration of the inventive, completed group of teeth, which have been manufactured by surfaces grinding,

FIG. 7 is an illustration of the completed, inventive toothing with teeth manufactured by surface grinding according to FIG. 6,

FIG. 8 is a schematic side view of an embodiment of the inventive saw blade according to a second embodiment,

FIG. 9 is an enlarged partial view of a first pair of the inventive teeth of FIG. 8,

FIG. 10 is a complete illustration of the structure of the inventive toothing according to the first embodiment by using the pair of teeth of FIG. 9,

FIG. 11 is an enlarged detailed illustration of an alternative embodiment of the toothing of FIG. 9,

FIG. 12 is an illustration of the complete toothing using the embodiment shown in FIG. 11,

FIG. 13 is an enlarged partial view of a further embodiment of the inventive toothing,

FIG. 14 is a complete illustration of the embodiment of the toothing according to FIG. 13, and

FIG. 15 is an illustration of a toothing having been modified by lateral channel grindings.

### DETAILED DESCRIPTION OF THE INVENTION

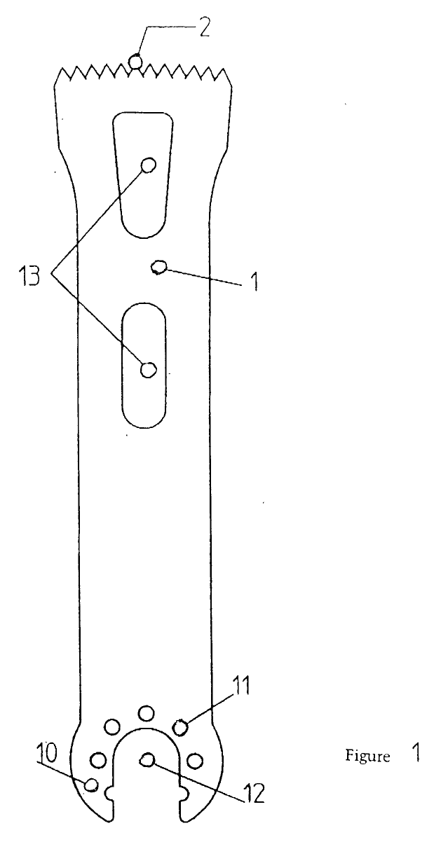

As can be seen in FIG. 1, the inventive saw blade comprises a substantially circular clamping portion 10, which is provided with a plurality of mounting openings 11, 12. Both the mounting openings and the dimension of the clamping portion 10 are known from the prior art, so that a detailed description can be dispensed with here.

The clamping portion is followed by the real body 1 of the saw blade, which is provided with a toothing 2 at its edge portion. The body 1 can be provided with a plurality of recesses 13.

Dimensions and arrangement of the flat, plate-like body 1 as well as of the equally flat, plate-like clamping portion 10 are known from the prior art and may be adapted according to the respective requirements. The toothing 2 preferably has an arcuate shape in order to obtain a regular cutting movement when oscillating about the center axis of the mounting opening 12.

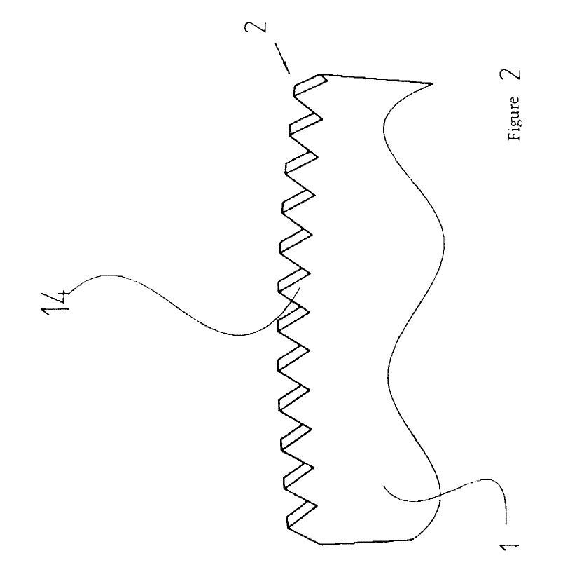

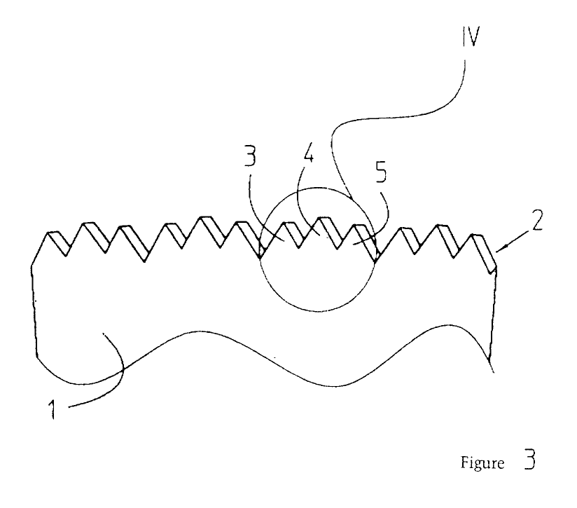

FIGS. 2 and 3 show detailed depictions of different manufacturing steps of the inventive toothing.

In a first processing step (FIG. 2), the base toothing is manufactured. The cutting edges of the single teeth 14 are hereby identical in geometry, the tooth is for example ground into the saw tooth blank by a grinding wheel in a determined angle (e.g. 60°). All cutting edges of the single teeth 14 therefore comprise cutting edges which are aligned obliquely to the front of the body 1.

FIG. 3 shows a manufacturing alternative in which the teeth are formed with different heights.

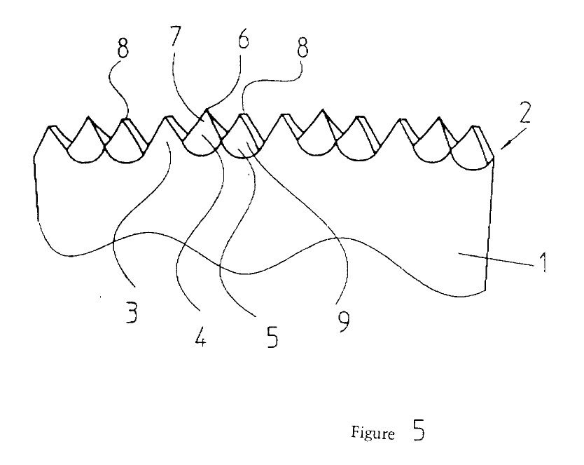

In a subsequent processing step, resulting from a comparison of FIGS. 2 and 3 with FIG. 5, the inventive channel grindings 7, 9 are generated.

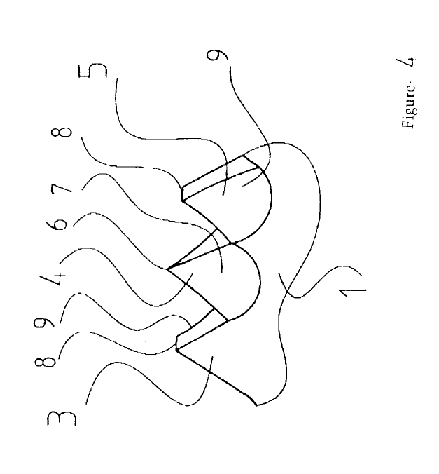

In FIG. 4, an enlarged depiction shows more clearly the three-teeth-structure of the inventive toothing. Said toothing each comprises lateral teeth 3 and 5 as well as an intermediate tooth 4. The lateral teeth 3 and 5 are provided with a channel grinding 9 in relation to the surface of the body 1, said channel grinding being formed only on one side of each tooth 3 or 5, respectively, and not extending beyond the center of the respective tooth in relation to the thickness of the originally produced tooth blank 14. Thus, the teeth 3, 5 are provided with cross cuttings 8 extending beyond the center plane (not shown) of the body 1.

The intermediate tooth 4 is provided with a channel grinding 7 on both sides, thus resulting in a lance 6.

The intermediate tooth 4 may be higher than the two lateral teeth 3, 5, such that the lance 6 exerts an additional centering effect.

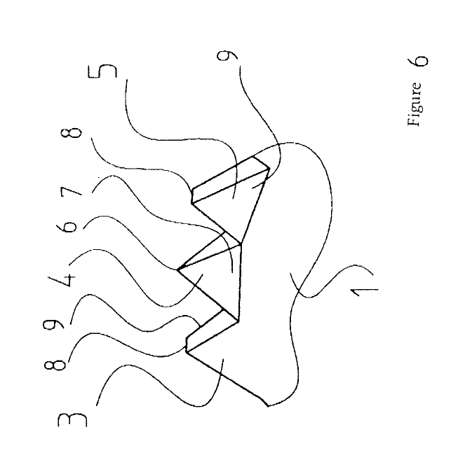

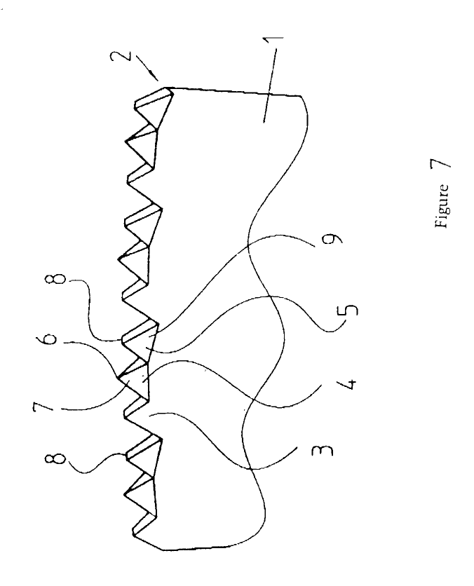

As an alternative to the generation of a channel grinding, the teeth may also be provided with ground surfaces, as shown in FIGS. 6 and 7. Such surfaces generated by surface grounding exhibit similar advantages as the channel grindings. Dependent on the geometries and the materials, however, additional advantages may arise with respect to the machining properties.

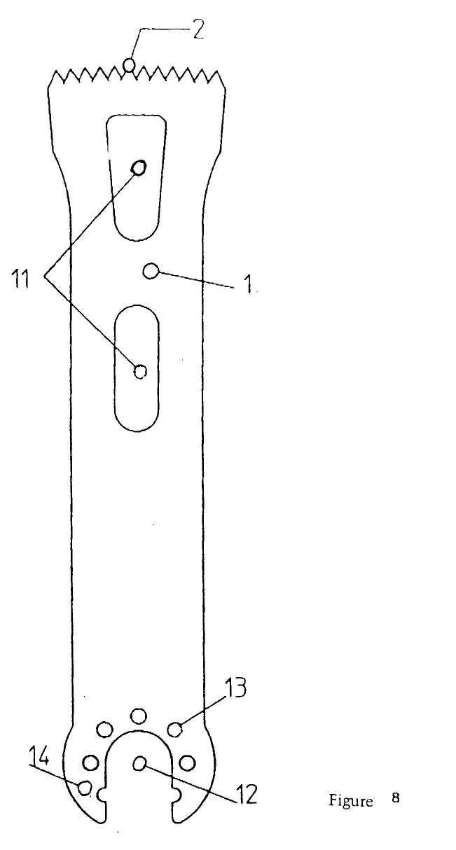

FIG. 8 shows a saw blade in side view, said saw blade comprising a substantially flat, plate-shaped body 1 which is provided with an arcuate toothing 2 at its edge portion. The body 1 is followed by a substantially circular clamping portion 14 being provided with mounting openings 12, 13. The dimensions and arrangements of the body 1 as well as the mounting openings 12 and 13 and the clamping portion 14 correspond to the prior art and may be adapted to the respective requirements. A detailed description at this place can therefore be dispensed with.

The toothing 2 preferably has an arcuate shape in order to obtain a regular cut during an oscillating reciprocating movement about the center axis of the mounting opening 12.

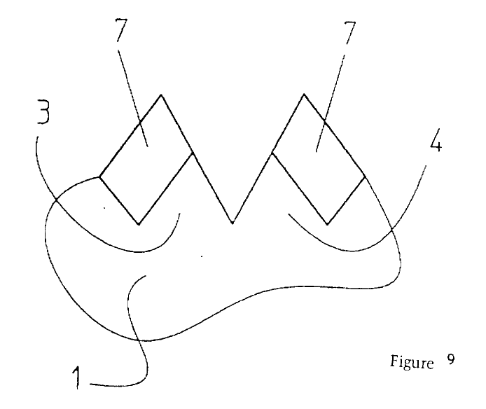

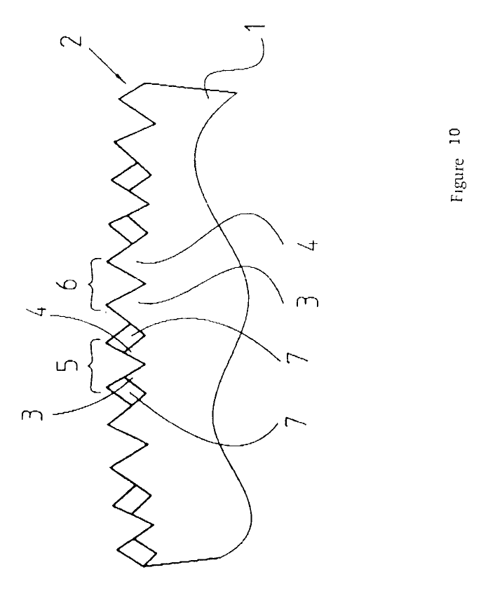

FIGS. 9 and 10 show an embodiment of the inventive toothing. Said toothing comprises pairs 5, 6 of two teeth 3, 4 each. FIG. 9 shows an enlarged detailed view of such a pair of teeth 3, 4. The teeth are arranged symmetrically to a symmetry axis being perpendicular to the center plane of the body 1. They are provided with a chamfer 7 respectively at the averted side, by which the corresponding cutting edges are formed. Both teeth of each pair 5, 6 are equally high and were substantially manufactured from a tooth blank (not shown) having the shape of an equal-sided or oblique-angled triangle.

As shown in FIG. 10, pairs 5, 6 of teeth 3, 4 are provided alternatingly. The pairs of teeth are identical, their orientation however is chosen alternatingly in relation to the (not shown) center plane of the body 1, such that the chamfers of the one pair 5 point to one side of the body 1, whereas the chamfers of the other pair 6 point to the other side of the body 1.

Due to the symmetric structure of the two teeth 3, 4 of a pair and due to the sequence of pairs being respectively arranged symmetrically to each other, a very regular cut is obtained with forces regularly exerted on the cutting area. Therewith, oscillations as well as deviations or de-centering of the saw blade during the cutting process are prevented.

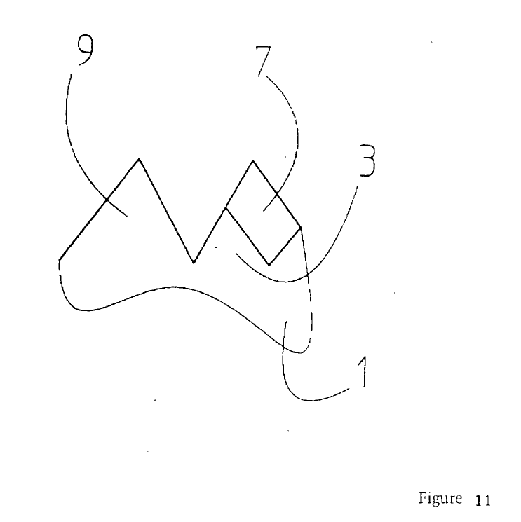

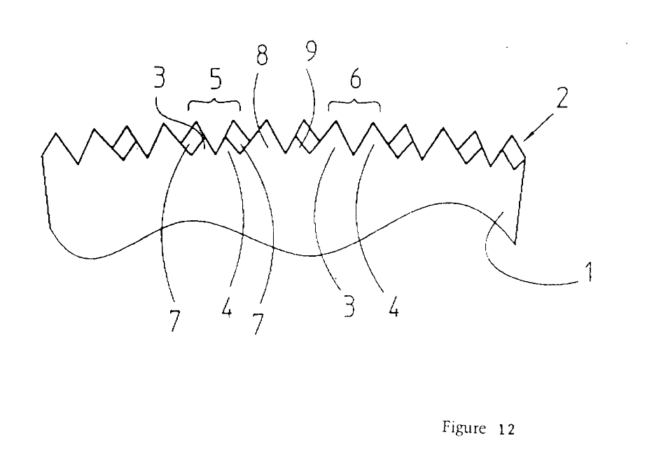

FIGS. 11 and 12 show an adapted embodiment in which additional teeth 8, 9 are arranged between each of the single pairs 5, 6. FIG. 11 shows an enlarged detailed depiction thereof.

As is especially discernible from FIG. 12, identical teeth are used in each case, also for the additional teeth 8, 9. The arrangement and orientation of the additional teeth 8, 9 is determined such that the additional tooth 8 following a pair 5 is identical to the first tooth 3 of the following pair 6. In the same way, the additional tooth 9 following tooth 3 of pair 6 is formed identical to the first tooth 4 of the following pair 5 of teeth.

The additional teeth 8, 9 may have the same height as the teeth 3, 4 of the respective pair 5 or 6. However, it is also possible that the additional teeth 8, 9 are higher or lower.

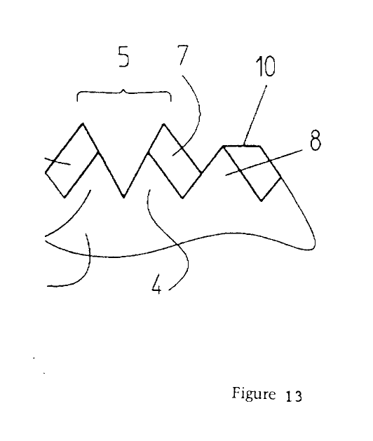

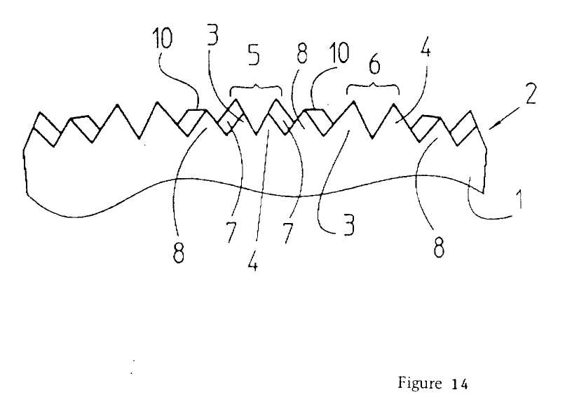

FIGS. 13 and 14 show a further embodiment in which only one additional tooth 8 is arranged between the adjacent pairs 5 and 6. This tooth has a different geometry. In the shown embodiment, the tooth is formed lower and is provided with a cross cutting 10, as may be seen also in the enlarged depiction in FIG. 13.

As already mentioned, the inventive toothing results in a precise saw cut being very efficient. The width of the cut corresponds at least to the size of the saw blade.

Especially in the embodiment shown in FIGS. 13 and 14, the arrangement of pairs 5, 6 of teeth 3, 4 results in a good centering, while the somewhat lower additional tooth 8, due to its cross cutting 10, contributes to a particularly efficient material removal.

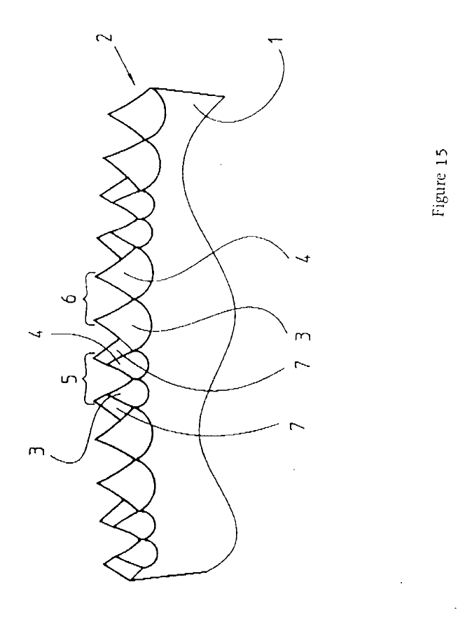

FIG. 15 shows an embodiment similar to that of FIG. 14, in which all teeth are provided with lateral channel grindings. It is clear that also single groups may be provided with such lateral channel grindings. It is also clear that the channel grindings do not have to be provided on both sides of the teeth, it is also possible to provide the single groups with a channel grinding only on one side, to arrange the channel grindings alternately or to chose an alternatingly embodiment.

Furthermore, it is possible within the scope of the invention to cross all teeth. It is clear that also single groups or single teeth can be crossed, while other groups or other teeth remain uncrossed.

The invention is not limited to the illustrated embodiments; rather, many alterations and modifications are possible within the scope of the present invention. The embodiments were chosen and described in order to best explain the principles of the invention and its practical application to thereby enable others skilled in the art to best utilize the invention in various embodiments and with various modifications as are suited to the particular use contemplated. As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

We claim:

1. A surgical saw blade having a flat, plate-shaped body being provided at least at an edge portion with a toothing comprising a plurality of uncrossed teeth, the teeth being respectively formed of groups, each group comprising three teeth of different shape.

2. The saw blade according to claim 1, wherein the intermediate tooth of the group comprises a lance.

3. The saw blade according to claim 1 or 2, wherein the intermediate tooth of the group is arranged symmetrically in relation to a center plane of the body.

4. The saw blade according to one of claims 1 to 3, wherein the intermediate tooth of the group comprises a channel grinding on both sides thereof.

5. The saw blade according to one of claims 1 to 4, wherein the two lateral teeth of the group each comprise a cross cutting.

6. The saw blade according to one of claims 1 to 5, wherein the two lateral teeth of the group comprise a channel grinding formed at one side flank.

7. The saw blade according to claim 6, wherein the channel grinding of the tooth is formed from a flank to the center plane in relation to the center plane of the body.

8. The saw blade according to one of claims 6 or 7, wherein the two lateral teeth are formed towards different sides of the saw blade with respect to the channel grinding.

9. The saw blade according to one of claims 1 to 8, wherein all three teeth are of substantially the same height.

10. The saw blade according to one of claims 1 to 8, wherein the height of the intermediate tooth is higher than the height of the two lateral teeth.

11. The saw blade according to one of claims 1 to 10, wherein the toothing is generated by surface grinding.

12. The saw blade according to one of claims 1 to 11, wherein at least one of the teeth are crossed.

13. A surgical saw blade having a flat, plate-shaped body being provided at least at an edge portion with a toothing comprising a plurality of uncrossed teeth, wherein the toothing respectively comprises pairs of two teeth which are arranged symmetrically to each other in relation to a plane perpendicular to a center plane of the body.

14. The saw blade according to claim 13, wherein each tooth has the shape of a body being formed by an equal-sided or oblique-angled triangle in side view, at one edge of which a chamfer is ground on one side.

15. The saw blade according to claim 14, wherein the chamfers of each pair of teeth are formed pointing away from each other.

16. The saw blade according to one of claims 13 to 15, wherein the pairs of teeth are each arranged alternatingly with respect to the center plane of the body.

17. The saw blade according to one of claims 13 to 16, further comprising at least one additional tooth arranged between two adjacent pairs of teeth.

18. The saw blade according to claim 17, wherein the at least one additional tooth is of substantially the same height as the teeth of the pair.

19. The saw blade according to claim 17, wherein the at least one additional tooth has a height different than the height of the teeth of the pair.

20. The saw blade according to claim 19, wherein the at least one additional tooth has a height greater than the height of the teeth of the pair.

21. The saw blade according to claim 19, wherein the at least one additional tooth has a height smaller than the height of the teeth of the pair.

22. The saw blade according to one of claims 17 to 21, wherein the at least one additional tooth comprises a cross cutting.

23. The saw blade according to one of claims 17 to 22, further comprising two additional teeth arranged between the two adjacent pairs of teeth, wherein the tooth following a pair of teeth is identical to the respectively following tooth of the next pair.

24. Saw blade according to one of claims 17 to 23, wherein single or all cutting edges are laterally modified by channel grindings.

25. The saw blade according to one of claims 18 to 24, wherein single or all cutting edges are crossed.
